(19) United States
(12) Patent Application Publication
(10) Pub. No.: US 2026/0364250 A1
(43) Pub. Date: Nov. 19, 2026

(54) USER-WEARABLE INFUSION PUMP SYSTEM

(71) Applicant: Tandem Diabetes Care, Inc., San Diego, CA (US)

(72) Inventors: Michael MICHAUD, San Diego, CA (US); Maxwell HUME, San Diego, CA (US); Grant GANGENESS, San Diego, CA (US); Ryan BETTS, San Diego, CA (US); Daniel MORRISON, San Diego, CA (US)

(73) Assignee: Tandem Diabetes Care, Inc., San Diego, CA (US)

(21) Appl. No.: 19/678,885

(22) Filed: May 15, 2026

Related U.S. Application Data
(60) Provisional application No. 63/806,155, filed on May 15, 2025.
(60) Provisional application No. 63/964,823, filed on Jan. 21, 2026.

(51) Int. Cl. A61M 5/142

(52) U.S. Cl. CPC A61M 5/14248; A61M 2005/14268; A61M 2202/07

(57) ABSTRACT

A tubeless patch pump system is disclosed herein. The pump can include a reusable drive unit and a single-use, disposable cartridge. The system can further include a tray for retaining the pump on the user's body and an inserter for applying the tray on the user's body and inserting a cannula for delivery of medicament from the pump through the tray and into the user's body. The pump includes specific design features that enable the pump to provide the same 200-unit medicament capacity as similar pumps in a smaller form factor.

### PRIORITY CLAIM

The present application claims the benefit of U.S. Provisional Application No. 63/806,155, filed May 15, 2025; and U.S. Provisional Application No. 63/964,823, filed Jan. 21, 2026, each of which is hereby incorporated herein by reference in its entirety.

### TECHNICAL FIELD

The present disclosure relates to medical pumps for delivering medicament to a patient and, more specifically, to a user-wearable pump, such as a patch pump, for delivering medicament such as insulin to a patient.

### BACKGROUND

There are many applications in academic, industrial, and medical fields that benefit from devices and methods that are capable of accurately and controllably delivering fluids, such as liquids and gases, that have a beneficial effect when administered in known and controlled quantities. Such devices and methods can be particularly useful in the medical field where treatments for many patients include the administration of a known amount of a substance at predetermined intervals.

One category of devices for delivering such fluids is that of pumps that have been developed for the administration of insulin and other medicaments for those suffering from both type I and type II diabetes. Some pumps configured as portable infusion devices can provide continuous subcutaneous medicament injection and/or infusion therapy for the treatment of diabetes. Such therapy may include, e.g., the regular and/or continuous injection or infusion of insulin into a person suffering from diabetes and offer an alternative to multiple daily injections of insulin by an insulin syringe or an insulin pen. Such pumps can be ambulatory/portable infusion pumps that are worn by the user and may use replaceable cartridges. Such pumps can deliver medicaments other than or in addition to insulin, such as glucagon. Examples of such pumps and various features that can be associated with such pumps include those disclosed in U.S. Patent Application Publication No. 2013/0053816, U.S. Pat. Nos. 8,573,027, 8,986,253, U.S. Patent Application Publication No. 2013/0324928, U.S. Patent Application Publication No. 2013/0331790 and U.S. Pat. No. 8,287,495, each of which is hereby incorporated herein by reference in its entirety.

One type of pump that has been developed is a patch pump, or micro pump. Patch pumps generally are small pumps, typically ambulatory, that are carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site. Patch pumps can be at least in part disposable, meant to be worn for a period of time such as, e.g., 1-3 days, and then discarded and replaced by a new patch pump. Other patch pump designs contemplate a disposable component, such as a cartridge that contains medicament, and a reusable or durable component. In such configurations, the disposable and durable components may be joined together by the patient or caregiver in preparation for delivery of the medicament.

### SUMMARY

A tubeless patch pump system is disclosed herein. The pump can include a reusable drive unit and a single-use, disposable cartridge. The system can further include a tray for retaining the pump on the user's body and an inserter for applying the tray on the user's body and inserting a cannula for delivery of medicament from the pump through the tray and into the user's body. The pump includes specific design features that enable the pump to provide the same 200-unit medicament capacity as similar pumps in a smaller form factor.

In embodiments, a patch pump system can include an adhesive patch configured to be adhered to a body of a user, a retention frame disposed on the adhesive patch and an infusion pump configured to be releasably inserted onto the retention frame and including a reservoir configured to contain a medicament. A portion of the reservoir that is visible when the infusion pump is inserted onto the retention frame can be opaque and a surface of the reservoir that is not visible when the infusion pump is inserted onto the retention frame can be transparent to enable viewing of an amount of the medicament in the reservoir.

In embodiments, a patch pump system can include an adhesive patch configured to be adhered to a body of a user, a retention frame disposed on the adhesive patch, the retention frame including an outer rim and an infusion pump configured to be releasably attached to the retention frame. The infusion pump can include a release button including one or more clips configured to nest with the outer rim of the retention frame when the infusion pump is attached to the retention frame. The release button can be configured to be pushed inwardly to disengage the one or more clips from the retention frame. The infusion pump can further include one or more snap elements configured to extend over the outer rim of the retention frame, the snap elements configured to maintain engagement of the infusion pump with the retention frame when the release button is pushed inwardly.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1C depict a patch pump system according to the present disclosure.

FIGS. 2A-2C depict a patch pump system according to the present disclosure.

FIGS. 3A-3B depict a cartridge pack for a patch pump system according to the present disclosure.

FIGS. 4A-4D depict a cartridge filling procedure for a patch pump system according to the present disclosure.

FIGS. 5A-5C depict a cartridge and drive unit of a patch pump system according to the present disclosure.

FIGS. 6A-6C depict an inserter/patch pack of a patch pump system according to the present disclosure.

FIGS. 7A-7C depict an inserter/patch assembly of a patch pump system according to the present disclosure.

FIGS. 8A-8B depict a patch pump system according to the present disclosure.

FIGS. 9A-9B depict a patch pump and a charger of a patch pump system according to the present disclosure.

FIGS. 10A-10E depict an exemplary drive mechanism for a patch pump system according to the disclosure.

FIGS. 11A-11B depict patch pumps systems according to the present disclosure.

FIG. 12 depicts a patch pump system according to the present disclosure.

FIGS. 13A-13D depict a patch pump system according to the present disclosure.

FIG. 14 depicts a portion of the patch pump system of FIGS. 13A-13D.

FIGS. 15A-15C depict aspects of a patch pump system according to the present disclosure.

FIG. 16 depicts patch pump systems according to the present disclosure.

FIGS. 17A-17D depict a cartridge filling system according to the present disclosure.

FIGS. 18A-18D depict a cartridge filling system according to the present disclosure.

FIG. 19A-19F depict a patch pump and a charger of a patch pump system according to the disclosure.

FIGS. 20A-20B depicts a charger for a patch pump system according to the disclosure.

FIG. 21 depicts a charger for a patch pump system according to the disclosure.

FIGS. 22A-22C depict an inserter/patch assembly of a patch pump system according to the present disclosure.

FIGS. 23A-23D depict aspects of a patch pump system according to the present disclosure.

FIGS. 24A-24F depict aspects of a patch pump system according to the present disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

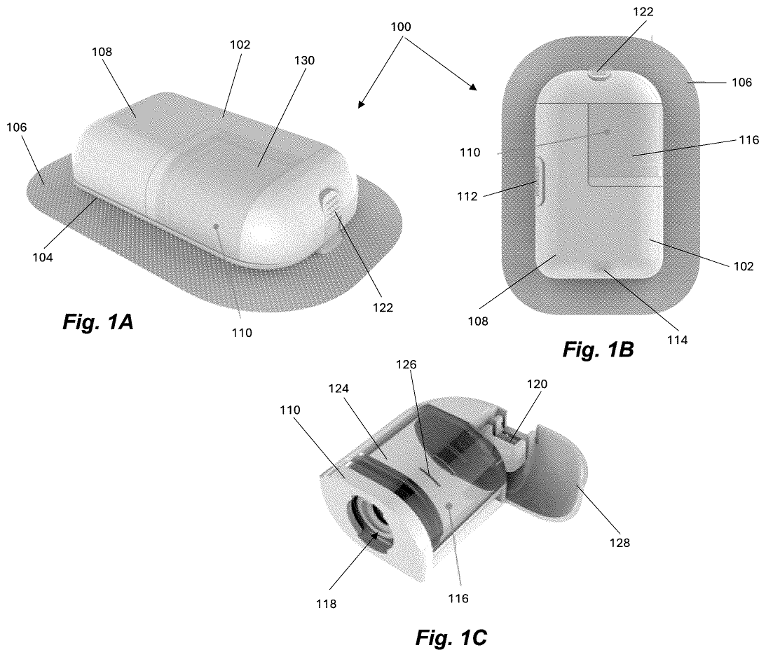

FIGS. 1A-1C depict a patch pump system 100 according to an embodiment of the disclosure. Patch pump system 100 can include a patch pump 102 selectively insertable onto a retention frame or tray 104. Tray 104 can be affixed to an adhesive patch 106 having an adhesive underside configured to attach the pump 102 and tray 104 to a body of a user. Pump 102 can include a drive unit 108 and a cartridge 110 selectively attachable to the drive unit. Pump 102 does not include a built-in display or user interface and is therefore primarily remote controlled. Various devices can be used to remotely control pump including, for example, a dedicated remote controller, a smartphone, a smartwatch, etc.

Drive unit 108 can be reusable and contain a drive mechanism that causes medicament in the cartridge 110 to be delivered to a user and other electronic components of the pump 102, such as, for example, a rechargeable battery, vibratory motor, speaker, etc. A function button 112 can be disposed on drive unit to enable various functions to be executed by the pump 102 without use of a remote control. Function button 112 may include a textured surface to aid a user in locating the button 112 without looking at the pump 102. In an embodiment, actuation of function button can cause a predetermined bolus amount of insulin to be delivered to the user. In some such embodiments, a varying amount can be delivered based on an amount of force used on the button (i.e., harder presses deliver larger boluses and softer presses deliver smaller boluses). Other functions that can be performed by button in various embodiment include silencing or snoozing (i.e., temporarily silencing for a predetermined period of time) various alarms (e.g., one click to snooze, two clicks to silence) or performing customized functions. For example, a mobile application for remote control of pump 102 could be programmed to customize various functions to be performed by the button, including different functions for different types of interactions with button (e.g., tapping, holding, double tapping, etc.).

Drive unit 108 can also include an indicator light 114 that can be illuminated in various ways to provide information to a user. In the depicted embodiment, pump 102 includes only a single indicator light 114, but the device could include two or more indicator lights. The pump can communicate with external devices (including for pairing) via various means through components contained within the drive unit 108, such as, for example, Bluetooth or Near Field Communications (NFC). In embodiments, drive unit housing can be formed from a polyimide material, such as, for example. polyetherimide (e.g. Ultem®). This material provides protection to device from damage from sunscreen, bug spray and other chemicals that a pump worn on a user's body may be exposed to. Other materials that can be used to form the housing include polyamide materials such as PA 12 or Nylon 12 or polycarbonate/polybutylene terephthalate. Such materials can be used with or without fillers like glass fibers, carbon fibers and glass beads.

Cartridge 110 defines a reservoir 116 configured to contain insulin or other medicaments. In various embodiments, cartridge 110 may be reusable and refillable or may be disposable after a single use (i.e., after the reservoir is emptied 116). Cartridge 110 can include an opening 118 designed to be inserted onto a portion of the drive unit 108 for attaching the cartridge 110 to the drive unit 108, as will be described in more detail below. Cartridge 110 further includes a cannula interface 120 that will interface with a cannula assembly to deliver medicament from the reservoir 116 through a cannula inserted into the user's body. A release button 122 can be disposed on an end of the cartridge 110 to aid in removing the cartridge from the tray 104. In embodiments, release button 122 can be provided with a textured surface. Reservoir 116 may be formed of a transparent material. An underside 124 of the reservoir may include graduated markings 126 such that a user can visually gauge an amount of medicament in the reservoir 116 and the transparent material enables the user to see any air bubbles that can be removed during filling. The upper surface 130 of reservoir 116 can be textured such that the transparent material becomes opaque, giving the upper surface of the pump 102 that is visible when the pump is worn by the user a more uniform appearance. In embodiments, the textured appearance can be achieved through various texturing processes of the mold for an injection molding process of the cartridge. An end cap 128 of cartridge 110 forms the entire upper, outwardly facing portion of pump 102 when the cartridge 110 is attached to the drive unit 108. The end cap 128 can be provided with various geometries to maintain a low profile of the pump for the user.

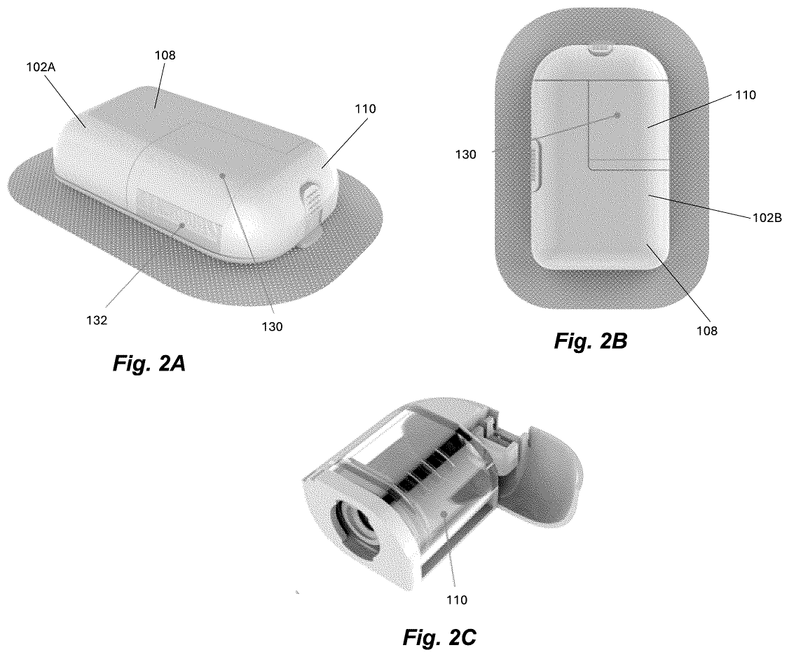

FIGS. 2A-2C depict a substantially similar patch pump 102A for a patch pump system including all of the features of patch pump 102 in FIGS. 1A-1C. In this embodiment, a top surface 130 of the cartridge 110 is completely opaque such that the appearance is identical to that of the drive unit 108. In embodiments, this top surface 130 can be pad printed. In such an embodiment, side surface 132 can be textured to provide opacity given the difficulty in pad printing paint onto curved surfaces. In various other embodiments surfaces 130, 132 can be painted by other means, comprise a piece of opaque plastic affixed to cartridge, etc.

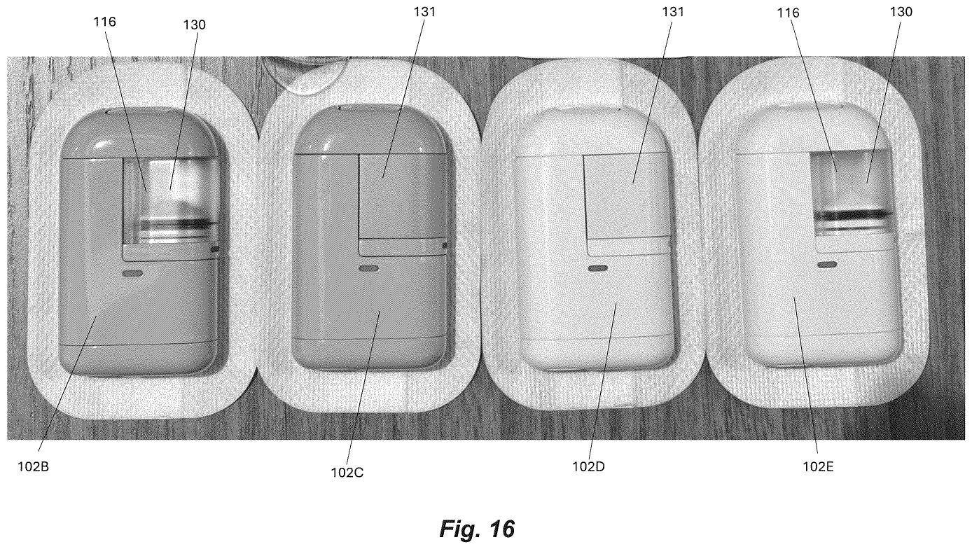

FIG. 16 depicts additional embodiments of a pump in which the top surface 130 of reservoir 116 may be partially see through (pumps 102B and 102E) or fully opaque (pumps 102C and 102D). In the latter embodiments, an adhesive sticker 131, which may have a color and texture matching that of the rest of the pump, can be affixed to the top surface 130 of reservoir 116. In some embodiments, such a sticker can be comprised of vinyl. In further embodiments, the color can be pad printed onto the top surface 130.

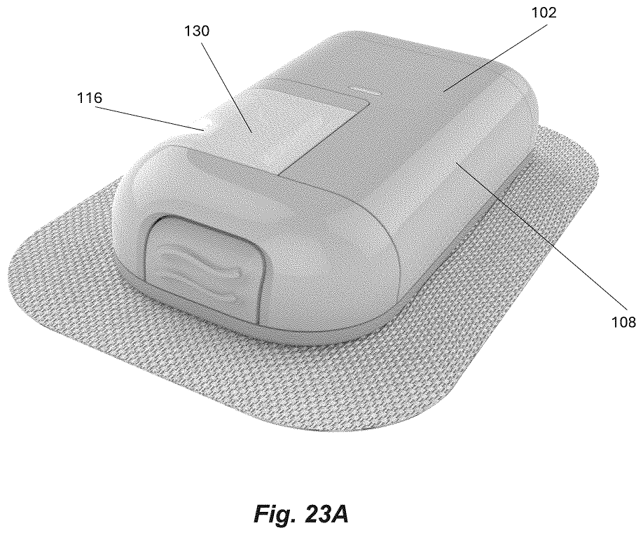

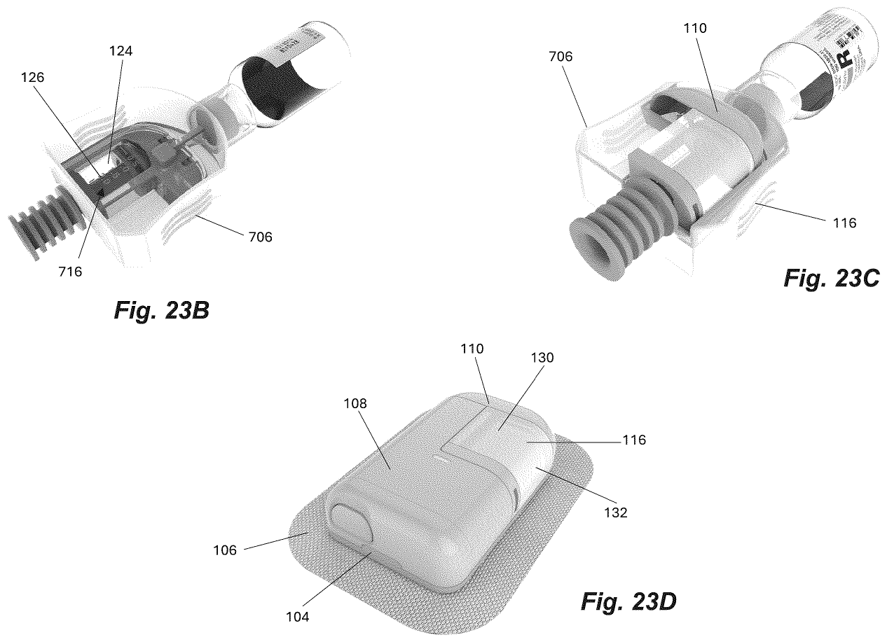

FIGS. 23A-23D depict another embodiment of the opacity approach described with respect to FIG. 16 that provides a monolithic outer appearance when the pump is worn by the user while having reduced opacity in an area of the cartridge viewed during filling of the reservoir with insulin. This approach is employed to minimize the medical-device aesthetic of the pump while preserving patient safety. Many pump users would prefer that the visual appearance of the pump not make it obvious that the user is wearing a medical device, and therefore the portion of the pump that is visible when worn by the user can be given a more uniform appearance that obscures the inner features of the cartridge that would most clearly identify the device as a medical device (e.g., the cartridge plunger within the reservoir). However, there are also important safety considerations that require a user to be able to visually observe the inside of the cartridge reservoir. The dual-opacity approach described herein accomplishes both functions.

For example, with reference to FIG. 23D when pump 102 is installed on the retention frame and worn on the body of the user with adhesive patch 106, only the top surface 130 and side surface 132 of the reservoir 116 of the cartridge 110 are visible. These surfaces can therefore be provided with a consistent color and opacity with drive unit 108 and other outer cartridge surfaces to provide a cohesive, uniform appearance that minimizes the visual prominence of the cartridge to blend more seamlessly with the overall design and provide less of a medical appearance. The bottom surface 124 of the reservoir can be less opaque, i.e., be clear enough to see through to enable a user to view the filling of medicament during the fill process through a viewing window 716 in a fill adapter 706 (described in more detail below). A user may be able to both gauge a level of medicament in the reservoir based on graduated markings 126 on bottom surface 124 and view whether any air bubbles exist in the medicament that should be removed.

In some embodiments, rather than achieving opacity through post-molding methods such as stickers or pad printing, a controlled combination of colorant and surface texture applied during the molding process achieves the dual optical functions required for both wear appearance and fill monitoring. For example, a colorant can be added to the naturally clear resin from which the reservoir is formed to achieve a desired degree of partial transparency. This partial transparency can then be enhanced on the bottom surface 124 and further obscured on the top surface 130 and side surface 132. For example, fill-volume markings 126 can be located on the bottom surface 124 and the external surface of the reservoir 116 in that area can be polished to maximize transparency for fill-volume monitoring and air-bubble detection during the filling procedure. Filling accessories, such as fill adapter 406 or fill adapter 706, can be designed to ensure that fill-volume markings 126 remain clearly visible to the user during the filling process. A matte texture can be applied to the surfaces 130, 132 of the reservoir 116 that are visible when the pump is attached to the infusion site to increase opacity of those surfaces and blend those surfaces with the surrounding drive unit and other cartridge surfaces. In some embodiments, the matte texture can be formed via texturing of the mold used in the injection molding process for the cartridge 110.

This dual-surface treatment allows reservoir 116 to serve dual optical functions providing the necessary transparency at bottom surface 124 for fill monitoring while presenting an opaque, uniform appearance consistent with the rest of the pump at the surfaces visible during normal wear. In some embodiments, rather than the top and side surfaces being opaque, they may be semi-transparent or otherwise at least partially opaque. In various other embodiments, both the top and rear of the cartridge can be provided with the same optical treatment, including both surfaces being fully transparent or partially transparent.

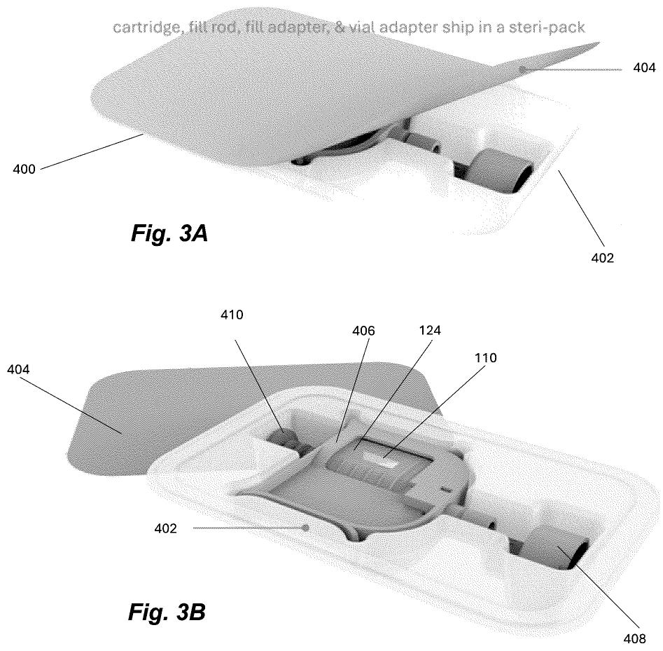

As noted above, cartridge 110 may be reusable and refillable or may be disposable after a single use. In the context of a disposable cartridge, each new (empty) cartridge 110 may be provided in a sealed, sterile cartridge pack 400 as depicted in FIGS. 3A-3B. In embodiments, cartridge 110 can be shipped with the transparent underside 124 of reservoir 116 facing upwardly. Cartridge pack 400 can comprise a plastic body 402 sealed with a paper seal 404. In addition to cartridge 110, cartridge pack 400 can contain a fill adapter 406, a vial adapter 408 and a fill rod 410 used to fill the cartridge 110 with medicament, as described below. Fill adapter 406 and vial adapter 408 can be separate components attachable to each other or can be unitarily formed as a single, monolithic construct. Although fill rod 610 id depicted as a generally cylindrical rod, other geometries are contemplated such as a rod having a circular handle with a circular aperture therein for grasping by a user. Paper seal 404 can be peeled off of plastic body 402 to provide access to these components when a new cartridge 110 is needed. The components may be packaged such that the cartridge 110 is seated in the fill adapter 406, the fill rod 410 is attached to the cartridge 110 and the vial adapter 408 is attached to the fill adapter 406 such that the components are already assembled when they are removed from the cartridge pack.

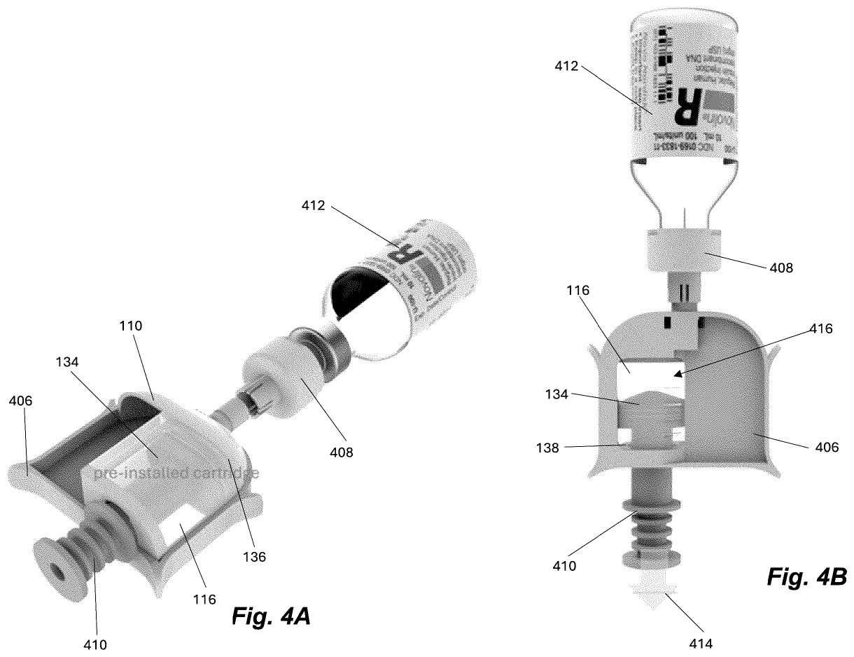

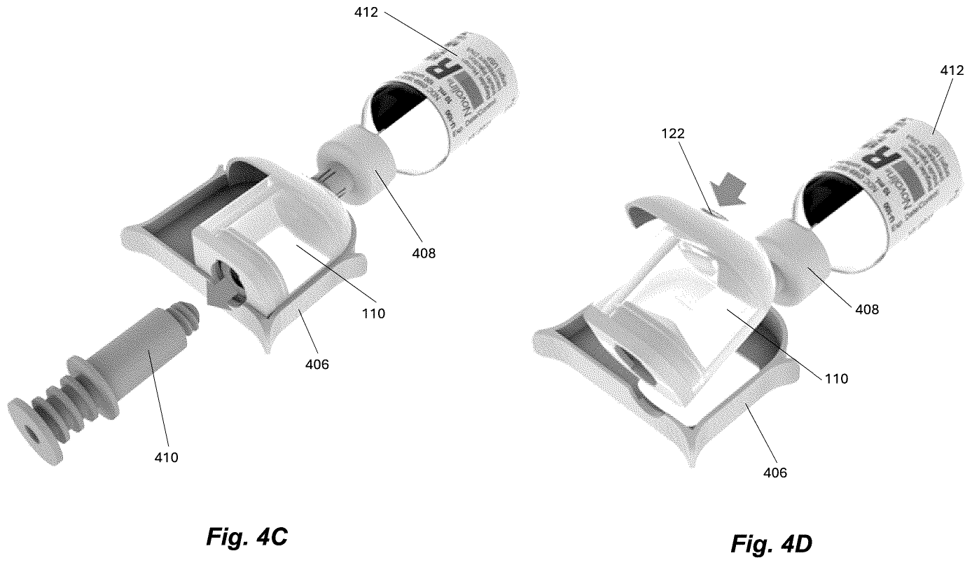

FIGS. 4A-4D depict a procedure for filling the cartridge 110 of the patch pump system of the disclosure with medicament. As noted above, the cartridge 110 may come out of a cartridge pack 400 pre-installed on the fill adapter 406 with the fill rod 410 attached thereto and the vial adapter 408 may be pre-attached to the fill adapter 406 (if they are separate components). A plunger 134 disposed within the reservoir 116 of the cartridge 110 is initially at a distal end 136 of the reservoir. A user then attaches an unused vial 412 of medicament to the vial adapter 408, which will puncture the vial and create a fluid communication path via a septum between the vial 412 and the reservoir 116. In embodiments, retention features (not pictured) within vial adapter 408 can secure the insulin vial 412 within the vial adapter 408 during the filling procedure. In some embodiments, the retention features can comprise bumps or projections that flex out of the way when the insulin vial 412 is inserted and then flex back to be secured around the insulin vial 412 around a neck portion of the vial beneath an upper rim of the vial.

To draw medicament from the vial 412 into the reservoir 116, the user pulls on the fill rod 410 in the direction of the arrow 414 in FIG. 4B until the plunger 134 reaches the proximal end 138 of the reservoir 116. The reservoir 116 is now filled to capacity with the medicament. Fill adapter 406 can include a viewing aperture 416 therethrough to enable a user to visually see the medicament being filled into the reservoir 116 from an under side of the fill adapter 406. If the user detects bubbles in the medicament in the reservoir 116 during filling, the user can manipulate the fill rod 410 until the bubbles are removed. After filling the reservoir 116, the fill rod 410 can be unscrewed from its attachment to the cartridge 110 and the release button 122 on cartridge 110 can be pressed to enable the cartridge 110 to be pivoted upwardly to disconnect it from the fill adapter 406. In other embodiments, rather than pivoting the cartridge 110 may be disconnected from the fill adapter 406 by linear upward movement. The fill adapter 406, vial adapter 408, fill rod 410 and vial 412 can then be discarded.

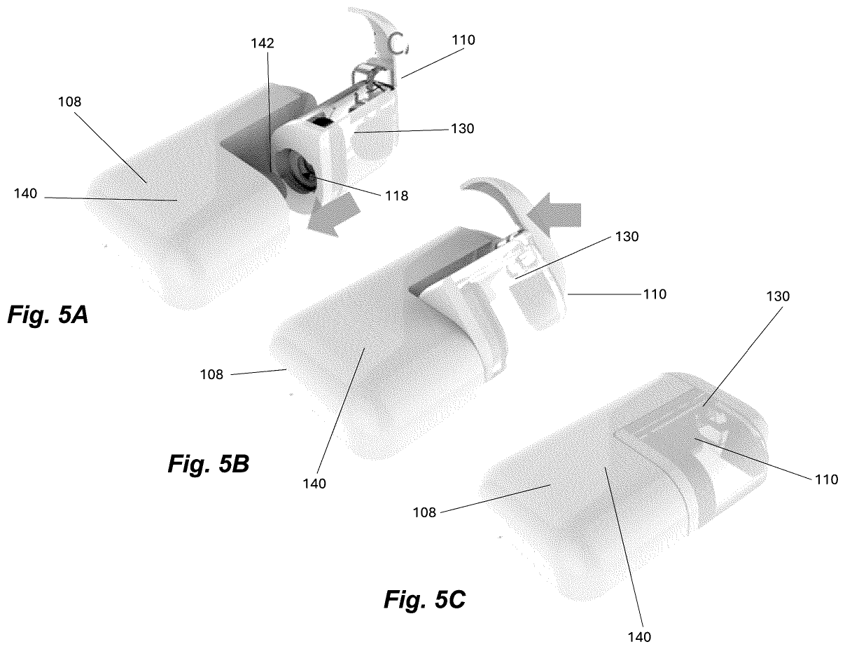

After filling the cartridge 110 with medicament as set forth in FIGS. 4A-4D, the cartridge is ready for use and can be attached to the drive unit 108. FIGS. 5A-5C depict one example method of attaching the cartridge 110 to the drive unit 108. Recess 118 of cartridge 110 can be configured to initially be inserted onto a drive mechanism 142 of drive unit 108 such that an outer surface 130 of the cartridge 110 is offset from an outer surface 140 of the drive unit 108 at an angle of, e.g., about 90 degrees as shown in FIG. 5A (generally perpendicular). The cartridge 110 can then be rotated toward the drive unit 118 to align the outer surface 130 of the cartridge 110 generally parallel with the outer surface 140 of the drive unit 108 and secure the cartridge 110 on the drive unit 108. In one embodiment, the engagement of the cartridge 110 to the drive unit 108 made by this rotation can cause an audible clicking sound that provides an auditory indication to the user that the cartridge is properly attached. Such a feature can alternatively or additionally provide a tactile indication to the user that the cartridge is properly attached. Although described above as an about 90 degree rotation, it should be understood that a wide variety of rotational angles can be employed, such as an angle of approximately 60 degrees, or any angle between about 30 degrees and about 150 degrees.

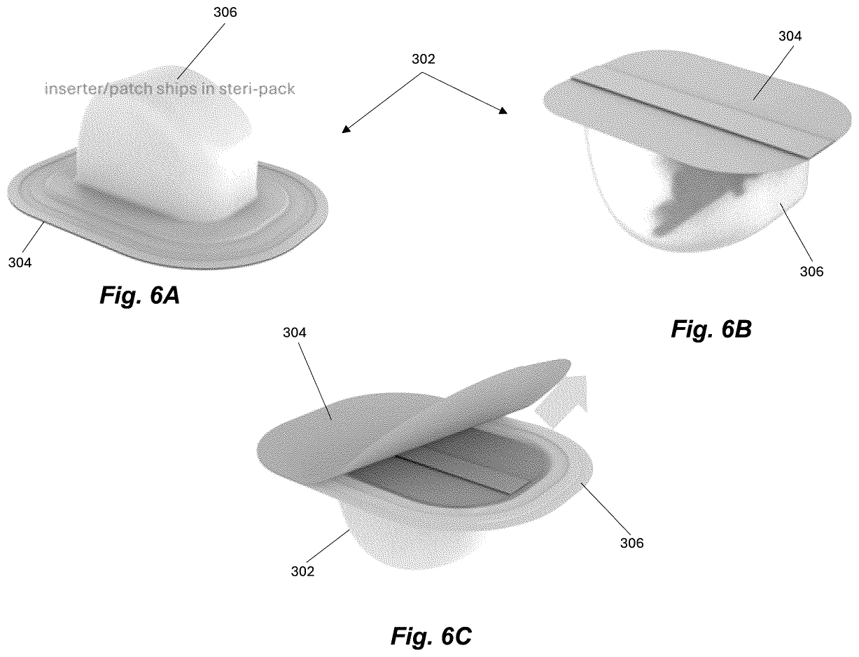

With the pump 102 now prepared for use, the user can attach the adhesive patch 106 and tray 104 to a desired location on the user's body. Tray 104, patch 106 and an inserter 300 can be packaged together in a sterile packaging 302 as shown in FIGS. 6A-6C. A paper backing 304 can be peeled off of the package housing 306 to provide access to these components. Once the components are removed from the packaging 302, the packaging can be discarded.

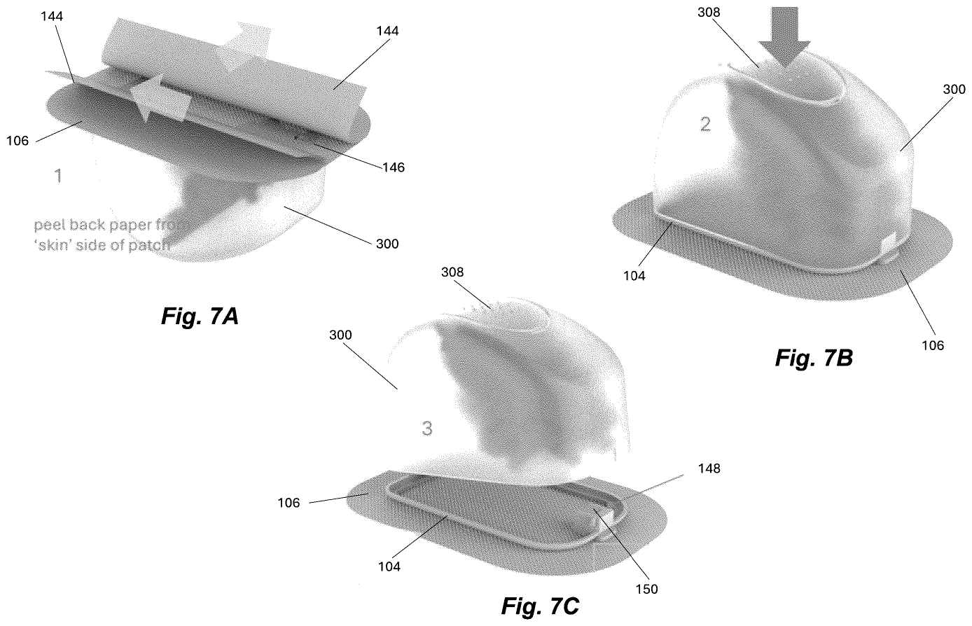

Referring now to FIGS. 7A-7C, when removed from packaging 302, the tray 104 is connected to the inserter 300 at a bottom of the inserter 300 with the adhesive patch 106 extending around the inserter 300. Initially, a user will peel off a backing 144 covering the adhesive 146 of the adhesive patch 106 as shown in FIG. 7A. The user will then place the adhesive patch 106 onto a desired location on the user's body. The inserter 300 can include an actuator button 308 that is actuated by pressing down on the button to simultaneously insert a cannula through a cannula port 148 in the tray 104 and into the user's body with a needle and release the tray 104 from the inserter. The inserter 300 can then be removed and discarded with the patch 106 retaining the tray 104 on the user's body with the cannula extending through the tray and into the user's body. A septum 150 in fluid communication with the cannula is seated in the cannula port 148 for fluidly connecting the pump 102 with the cannula.

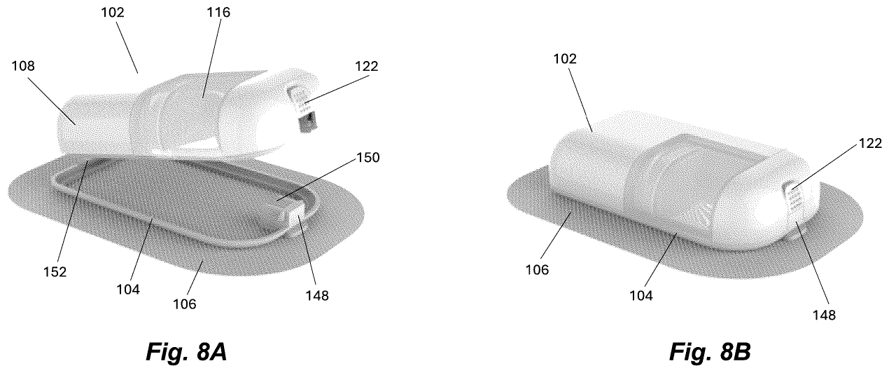

The pump 102 can now be attached to the tray 104. Referring to FIG. 8A, in one embodiment an aperture (not pictured) in a bottom end of drive unit 108 of pump 102 is inserted onto a hook 152 of tray 104 with the pump 102 at an angle. The pump 102 is then pivoted down to enable the front end of pump to snap fit onto cannula port 148 of tray 104. This snap fit may provide an audible clicking sound that indicates proper attachment to the user. This connection also causes a transfer needle (not pictured) in cartridge 110 in fluid communication with the reservoir 116 to penetrate the septum 150 in the cannula port 148 to establish fluid communication between the reservoir 116 and the cannula through the septum 150 in cannula port 148. The device is now ready to be operated to deliver medicament to the user. Pump 102 can be removed from tray 104 with the aid of release button 122 to disengage the snap fit between the pump 102 and the cannula port 148. In embodiments, there is a single clip on the front end of pump that snaps into a corresponding recess on the cannula port 148.

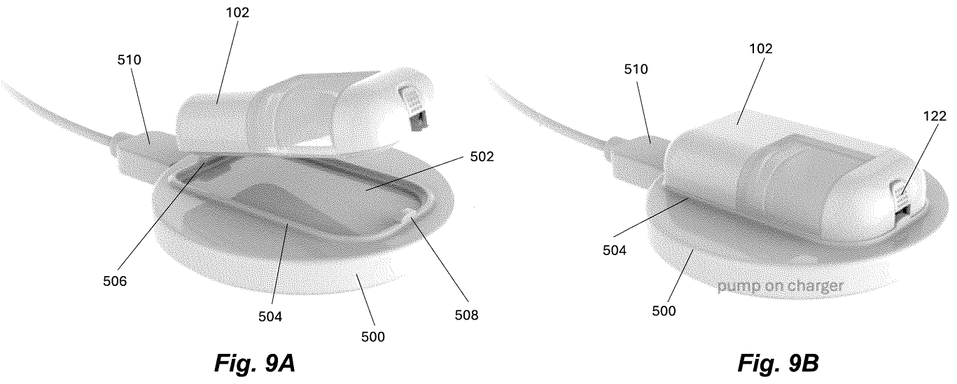

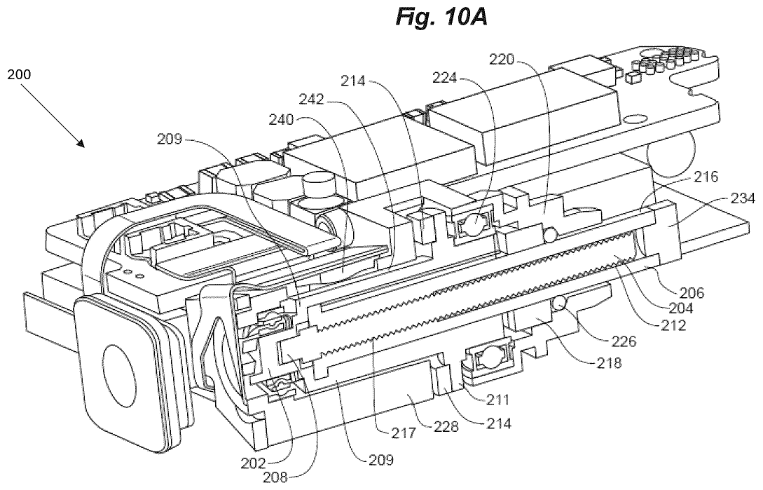

As noted above, pump 102 may include a rechargeable battery. In embodiments, the battery may be rechargeable via inductive charging. For example, system 100 may further include an inductive charging pad 500 connectable to a power outlet with a power cord 510 as depicted in FIGS. 9A-9B. Charging pad 500 may define a recess 502 and an annular rim 504 having a shape generally matching that of the pump 500 to guide proper positioning of the pump on the pad 500. Charging pad 500 can further include similar latching components to tray 104 to releasably hold the pump 102 on the charging pad 500. In particular, charging pad 500 can include a hook 506 onto which the aperture in the bottom end of drive unit 108 is inserted and a corresponding latch 508 on the opposite end that provides a snap fit with the top end of the pump 102. Release button 122 on pump 102 can be depressed to remove the pump 102 from the charging pad 500. In other embodiments, the battery of pump 102 can be recharged by other means, such as, for example, NFC.

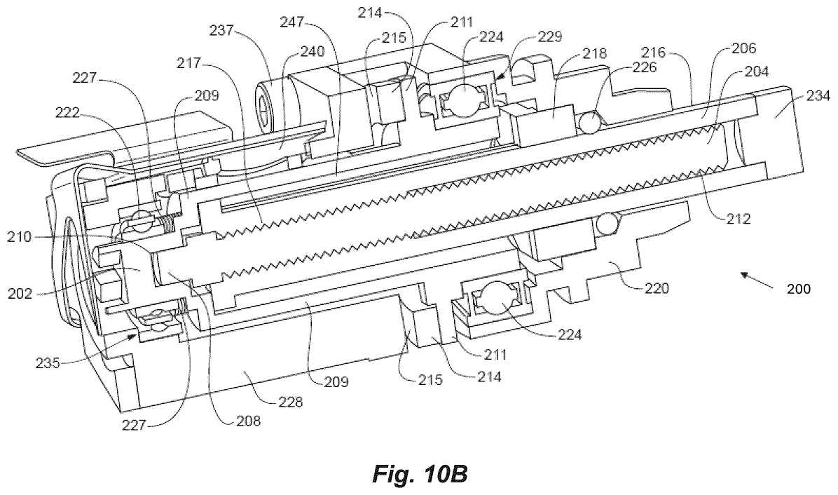

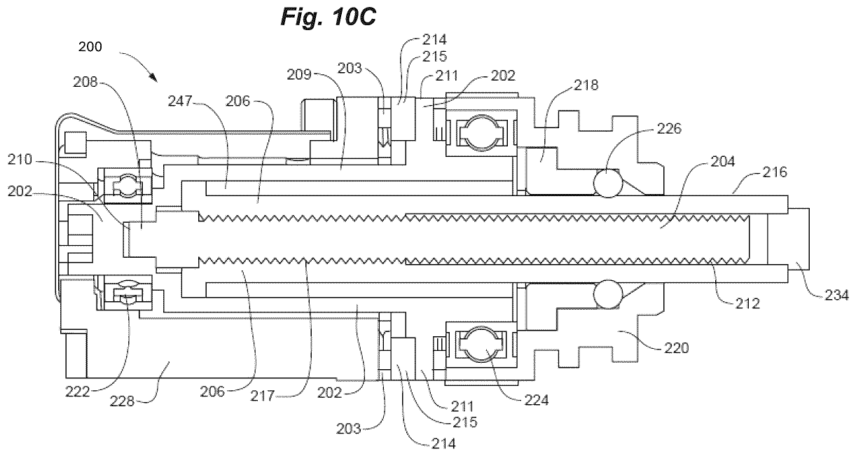

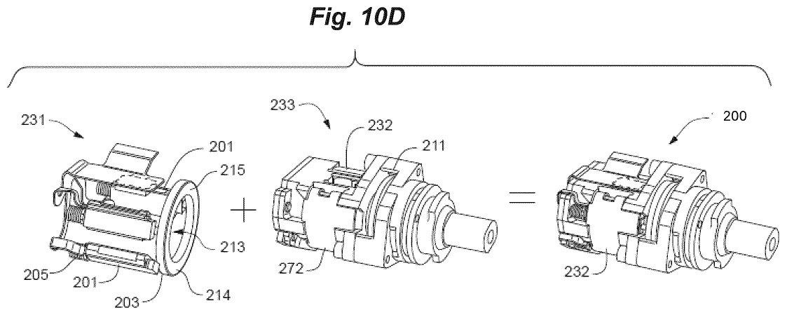

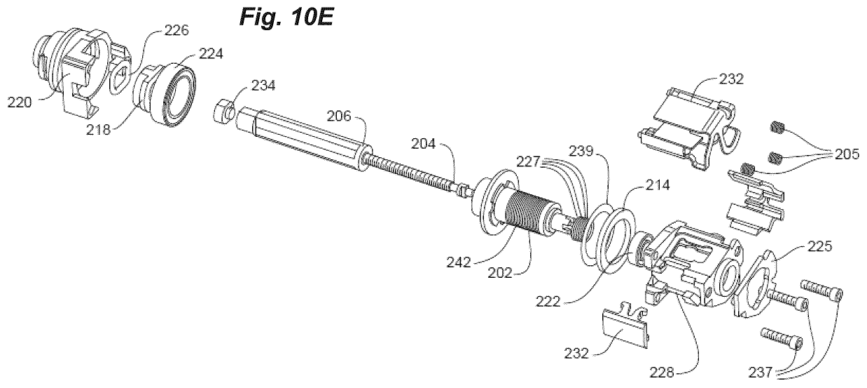

One exemplary drive mechanism 200 that can be housed within the drive unit 108 of pump 102 is depicted in FIGS. 10A-10E. Drive mechanism 200 utilizes piezoelectricity to dispense medicament from the cartridge 110. In general, drive mechanism 200 includes a plurality of piezoelectric elements that when energized with a drive signal oscillate against a drive disk to induce rotational motion of the drive disk. The rotational motion of the drive disk is converted to linear motion of a pushrod that interfaces with the syringe in the cartridge to advance the syringe and dispense medicament.

In the depicted embodiment, the piezoelectric elements that drive the system are three piezoelectric crystal motors 201 contained within the drive mechanism 122, although it should be understood that greater or fewer numbers of motors could be used. One type of piezoelectric motor that may be used in various embodiments of the present invention is the Edge Motor sold by Nanomotion Ltd. of Yokneam, Israel, a Johnson Electric Company. Motors 201 can be arranged in separate housings 207 circumferentially positioned around the drive mechanism 200 and each can include a drive tip 203 at one end that contacts the drive disk, such as ceramic drive disk 214, along a body portion 215 of the ceramic drive disk 214. Drive disk 214 can be configured, for example as shown in FIGS. 10A-10E, as an open ring. A coil spring 205 can be positioned at an opposite end of each motor 201 and be biased to ensure that the drive tips 203 of the motors 201 maintain contact with the ceramic drive disk 214 with the appropriate amount of force. In the depicted embodiment, the coil springs 205 can be retained by a spring retention end cap 225 and the bias can be provided to the coil springs 205 by a rear bearing 222, which can include a plurality of set screws or a plurality of shim washers 227, as shown in FIGS. 10B and 10E. Set screws or shim washers 227 can be used to adjust the extent of the preload provided to the coil springs 205 by the rear bearing 222. In embodiments, there are three set screws or five shim washers 227. Alternatively, in place of set screws or shim washers providing the preload bias, a threaded cap, wave spring, or elastomeric spring (o-ring), or any combination thereof, could be employed. Drive mechanism 200 can further include one or more retention clips 232 that aid in retaining the piezoelectric drive components 231 on a base portion 233 of drive unit 118 (see FIGS. 10D and 10E). The depicted embodiment includes two such clips 232.

The primary elements of the motor assembly utilized to translate the rotational motion of the ceramic drive disk 214 induced by the motors 201 into linear motion include a drive tube 202, a lead screw 204 and a drive nut or pushrod 206. As can be most clearly seen in FIGS. 10A-10C, the ceramic drive disk 214 is coupled to the drive tube 202 with a body 209 of the drive tube 202 extending through an opening 213 in the ceramic drive disk 214 and the body 215 of the ceramic drive disk 214 abutting a flange 211 of the drive tube 202. This interconnection of the ceramic drive disk 214 and drive tube 202 causes rotation of the ceramic drive disk 214 to induce rotation of the drive tube 202. In one embodiment, the ceramic drive disk 214 and drive tube 202 are attached to each other with a pressure sensitive adhesive 239 combined with an epoxy (See FIG. 10E). Alternatively, these components can physically interlock with each other such as, for example, with a key feature, hex, square, splines, polygon shape, etc. A proximal end 208 of the lead screw 204 is received within and coupled to a recess 210 in the drive tube 202, such that rotation of the drive tube 202 also rotates the lead screw 204. An internally threaded portion 217 of the pushrod 206 is coupled with external threads 212 of the lead screw 204. The external perimeter 216 of the pushrod 206 can be non-circular and slidably mated with a guide bushing 218 coupled to and contained within a front housing 220 to prevent the push rod 206 from rotating. This in turn enables rotation of the lead screw 204 to effect linear motion of the push rod 206. The push rod 206 is therefore contained within and directly driven by the motor assembly. A drive tip 234 can be attached to a distal end of the pushrod 206.

Various other components can be included in drive mechanism 200. Screws 237 extend from rear housing 228 to front housing 220 to retain device components together. Rear bearing 222 and a front bearing 224 can interface with the drive tube 202. A front seal 226 is provided between the front bearing 224 and guide bushing 218 to seal the interior of the drive mechanism 200. An additional sliding seal 247 that slides along the push rod 206 as it is advanced from the drive mechanism 200 can be included to provide redundant seals that prevent water and other liquid and/or contaminants such as dust, etc. from leaking into the system. Drive mechanism 200 can also include a flexible printed circuit board 230 that controls operation of the unit. In some embodiments, all electrical connections for each of the motors 201 can be contained on this flexible circuit board 230.

In one embodiment, operation of the unit is accomplished by energizing the piezoelectric motors with an electrical drive signal to cause them to oscillate against the ceramic drive disk 214. This oscillation induces rotational motion of the ceramic drive disk 214. Rotational motion of the ceramic drive disk 214 causes the drive tube 202 and the lead screw 204 to rotate. The intermeshing threaded exterior portion 212 of the lead screw 204 and interior threaded portion 217 of the pushrod 206 along with the non-circular perimeter 216 of the pushrod 206 that is constrained from rotating by the guide bushing 218 cause this rotational motion to be converted into linear motion of the pushrod 206. When the drive mechanism 200 is attached to a cartridge containing medicament, such as cartridge 116, the linear motion of the pushrod 206 causes the drive tip 234 of the pushrod to advance a syringe in the cartridge to cause medicament to be dispensed from the cartridge. Motors can be driven in parallel such that if one of the motors were turned off or not receiving the electrical drive signal, the non-energized motor would act as a friction brake due to the spring loading of the motor against the ceramic drive disk. The other two motors would continue to operate such that the system could still be in use with a non-functioning motor, however, the torque output would be significantly reduced relative to three operating motors. In other embodiments, greater or fewer than three motors can be employed, such as, for example two motors, with the number of motors affecting the amount of torque provided.

In some embodiments, drive mechanism 200 can further include a force transducer used to determine if the mechanism is functioning properly when activated to dispense medicament. Referring to FIG. 10B, in some embodiments, a force transducer can be located between the front bearing 224 and the front housing 220, as indicated, for example, by location 229. In such embodiments, axial force on the lead screw 204 when the device is operated will be transferred through the drive tube 202 such that force on the transducer 229 will decrease under a drive load. In other embodiments, a force transducer can be disposed between the rear bearing 222 and the rear housing 228 as indicated, for example, by location 235 in FIG. 10B. In such embodiments, axial force on the lead screw 204 during operation will be transferred through the rear bearing 222 to the transducer such that the force on the transducer 235 will increase under a drive load. In either type of embodiment, the respective decrease or increase in force will indicate that the device is operating properly, whereas the lack of decrease or increase will indicate that the device is not operating properly, such as when there is an occlusion preventing medicament from being dispensed from the cartridge. One or more of transducer 229 and/or transducer 235 can therefore be used as a means for detecting occlusions in patch pump.

There are a number of benefits provided by a patch pump such as described herein utilizing a piezoelectric drive mechanism as opposed to use of a conventional electromagnetic motor as in most current patch pumps. A piezoelectric drive unit can be made smaller than an electromagnetic motor of the same capability and typically has fewer moving parts because no gear reduction is needed to generate the force required to drive the syringe to dispense medicament from the cartridge. In addition, a piezoelectric motor is capable of effecting micron-sized increments of motion that in turn allow for precise and small movements of the syringe without the need for a gear reduction system. A piezoelectric motor also can afford greater safety when acting to dispense medicaments such as, e.g., insulin, because such a motor can automatically brake the drive system when turned off or deactivated. This in turn can prevent the unintended dispensing of medicament. This braking effect is due in part to the friction that exists between the piezoelectric drive tip(s) and the ceramic drive disk. Another safety concern for syringe drive systems related to unintended motor movement is addressed with the system of the present invention because the piezoelectric motors require a specific AC drive signal, such that a short circuit in the system to a DC voltage source could not result in unintended motor movement.

Because no gear reduction or gear transmission system is needed in embodiments of the present invention, the pushrod utilized to drive the syringe can be contained within and connected directly to the motor assembly. This “hollow motor” that is capable of containing the drive element for the system enables a syringe-type pump as described herein to be provided in a smaller size than has heretofore been possible with prior art electromagnetic-based syringe pumps and other pumps that utilized gear reduction systems.

Further details regarding such a drive mechanism and pumps employing such drive mechanisms can be found in U.S. Pat. No. 10,279,107 (the “'107 Patent”), which is hereby incorporated herein by reference in its entirety. The pump 102 disclosed herein includes certain modifications relative to the pump disclosed in the '107 Patent that provide particular enhancements and enable pump 102 to be provided in a smaller overall form factor while still having the same medicament capacity (e.g., 200 units). For example, one pump that has been developed according to the '107 Patent has length, width and height dimensions of 2.41″×1.59″×0.61″. Pumps developed according to the disclosure of the present application can have dimensions of 2.21-2.54″×1.32-1.52″×0.54-0.57″. This can be achieved by miniaturing the electronics within the drive unit, modifying the shape of the battery within the drive unit and utilizing a more volumetrically efficient cartridge

Miniaturizing the electronics in the drive unit can include using a flexible Printed Circuit Board Assembly (PCBA) such that the flexible PCBA can be folded around other components in the drive unit and providing a custom Bluetooth antenna (or other communication mechanism such as NFC) designed to fit within the available space within drive unit. The battery shape can also be customized based on the available space within the drive unit to fit around the other components within the drive unit.

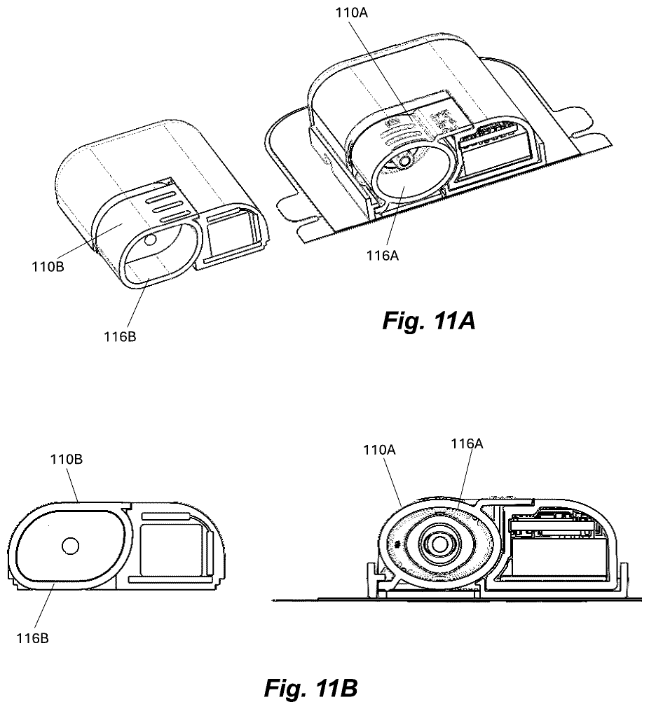

FIGS. 11A-11B depict side by side views of a previously developed cartridge 110A having a reservoir 116A (and corresponding syringe plunger (not pictured) of a first shape and a cartridge 110B according to the present disclosure having a reservoir 116B (and plunger) of a different shape. As can be seen in these figures, whereas reservoir 116A has an elliptical shape, the shape of reservoir 116B is a parallelogram-like shape with rounded corners. This shape of reservoir 116B has been found to be more volumetrically efficient while still maintaining the same 200 unit medicament capacity, enabling the device to have an overall smaller footprint as can be seen in FIGS. 11A-B.

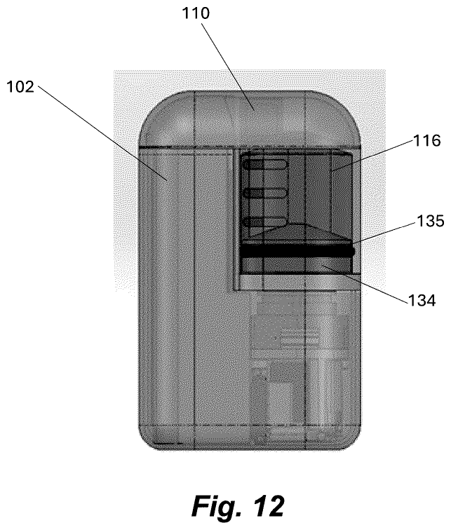

Pumps as disclosed herein can also utilize an improved combination of materials that aids in sterilization and device performance. Typical infusion pump reservoirs are sterilized using ethylene oxide (EtO), but EtO can cause health and environmental risks and the process can be relatively expensive. The materials can be selected for pump such that sterilization can instead be performed by radiation while maintaining drug compatibility and stick-slip performance. In particular, the reservoir 116 of the cartridge can be formed of a copolyester material and an o-ring 135 around syringe 134 (see FIG. 12) that interfaces with the internal surfaces of the reservoir 116 can be formed from Ethylene Propylene Diene Monomer (EPDM). One exemplary EPDM o-ring is provided by Parker-Hannifin, which is internally lubricated (i.e., a lubricating agent is introduced into the compound during the mixing stage such that the lubricant is dispersed throughout the compound). This material combination can be sterilized via electronic beam radiation while minimizing variations in friction between the two components that lead to the stick-slip phenomenon that can cause a lack of the necessary precision in medicament delivery. This combination can also enable cartridge 110 to employ only a single o-ring 135 around plunger 134 while maintaining an effective seal. Plunger can be formed from any medical grade engineering plastic, such as, for example, polycarbonate, polyethylene, polypropylene, etc.

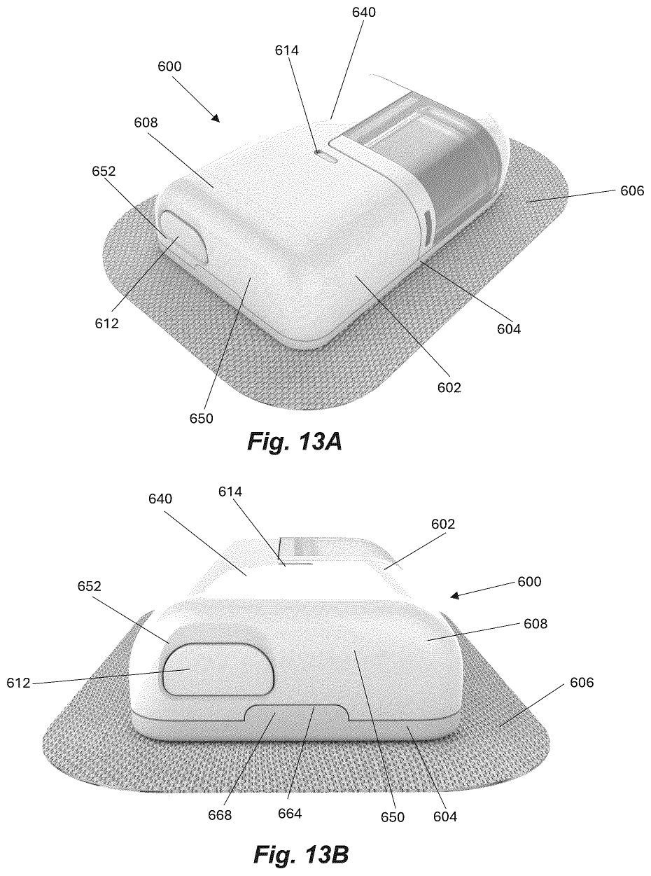

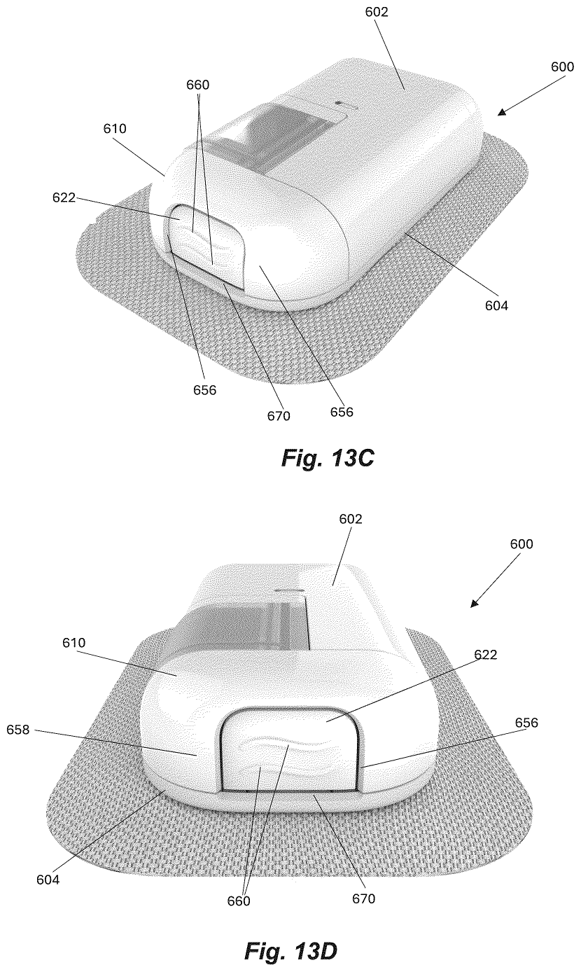

A patch pump system 600 according to another embodiment of the disclosure is depicted in FIGS. 13A-13D. Patch pump system 600 is similar to patch pump system 100 described above and includes a patch pump 602 and a tray 604 affixed to an adhesive patch 606 to attach the pump 602 to a body of a user.

In this embodiment of patch pump 602, the location of the function button 612 and the indicator light 614 are in different locations than the corresponding components of pump 102. The indicator light 612 is more centrally located on the top surface 640 of the drive unit 608. This makes it easier for the user to see the indicator light 612 when the pump is on the charger and enables a wider viewing angle of the light 612 when being worn by the user regardless of the orientation in which the system 600 has been placed on the user's body. The button 612 is located on an end of the drive unit 608 rather than on the side of the drive unit. This can be done for familiarity with other similar devices or based on constraints on the arrangement of hardware within the device. The button 612 is also positioned slightly inwardly from the outer surface 650 of the drive unit 608 within a recess 652 formed in the outer surface 650 of drive unit 608, which reduces the possibility of inadvertent activation of button 612.

The configuration of the release button 622 and its corresponding attachment to tray 604 has also been modified in the embodiment of FIGS. 13A-13D. Release button 622 is recessed within a shroud 656 in the end 658 of cartridge 610, which aids in decreasing the possibility of inadvertent actuation that could cause the pump 602 to unclip from the tray 604. Release button 622 also includes a pair of projections 660 configured as waves that provide gripping surfaces and can indicate to a user that the user is touching the release button 622 (e.g. the user reaches for the button 622 without looking at the pump 602). In the depicted embodiment, the waves are tapered at each end and positioned higher on the release button 622 in the middle of the button 622.

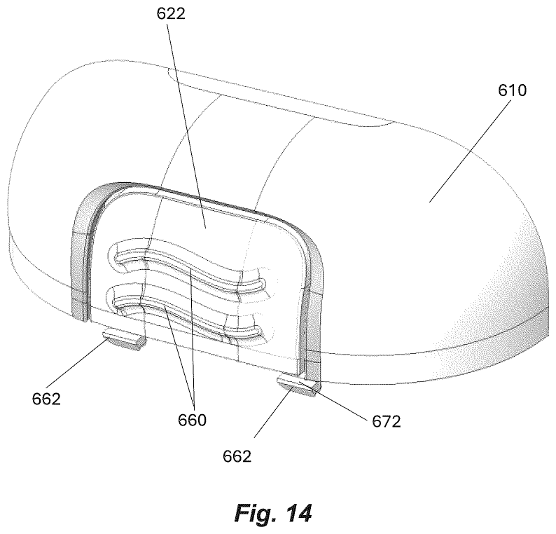

The partial view of the cartridge 610 in FIG. 14 depicts the clips 662 on button 622 that interface with the tray 604 to releasably lock the pump 602 onto the tray 604. Pump 602 is attached to tray 604 in a manner similar to that depicted in FIGS. 8A-8B with an opening 664 on the end 650 of drive unit 608 first inserted onto a hook 668 on the corresponding end of the tray 604, and the pump 602 then pivoted downwardly to interface the release button 622 and clips 662 with the tray. In particular, clips 662 will contact outer rim 670 of tray 602 causing the release button 622 and clips 662 to deflect inwardly. Once the clips 662 have cleared the outer rim 670, the release button 622 will automatically flex back outwardly to nest the clips 662 in a recess beneath the outer rim 670 with planar upwardly facing surface 672 of each clip 662 interfacing with a corresponding downwardly facing surface defined by recess beneath outer rim 670 to releasably lock the pump 602 on the tray 604. The pump 602 can be removed by pressing on the release button 622 to remove the clips 662 from the recess to disengage the locking surfaces 672 from the corresponding surfaces of the tray 604.

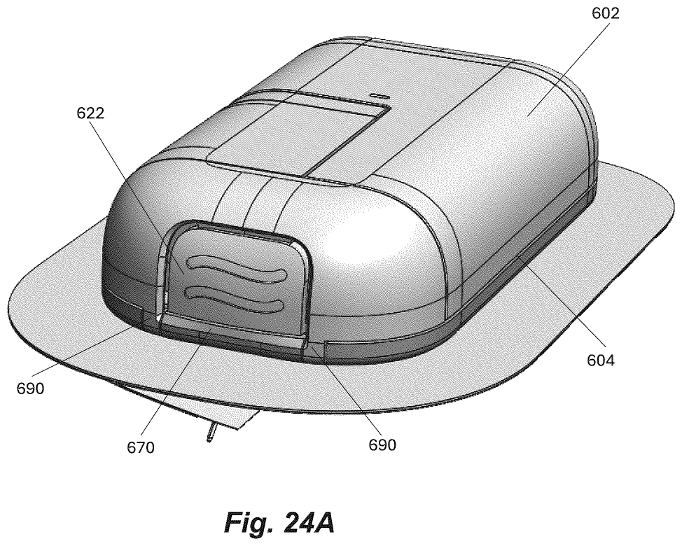

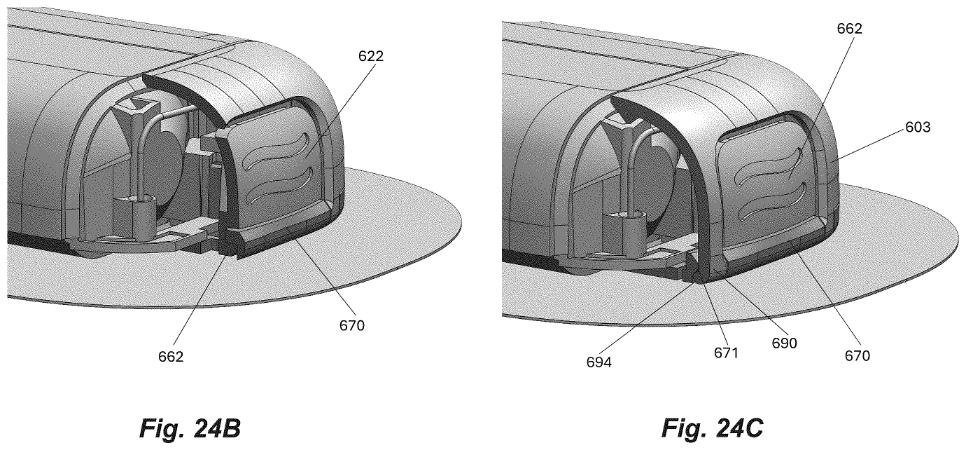

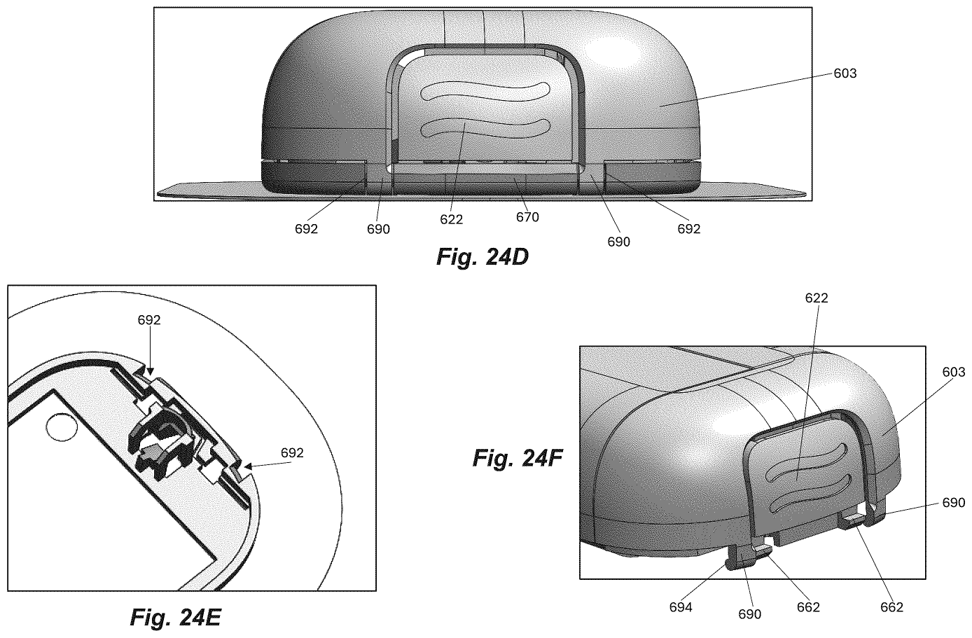

In some embodiments, the pump-to-tray retention mechanism can include a redundant retention system to reduce the likelihood of unintended pump disengagement during normal use. Accidental actuation of the primary retention latch—such as release button 622 with clips 662 may occur when the pump 602 is contacted by bedding, walls, or other surrounding surfaces during normal handling. To address this issue, a redundant retention feature can be provided as depicted in FIGS. 24A-24F.

For example, in embodiments a redundant retention feature can comprise passive snap elements 690 positioned on the sides of the primary latch, flanking the release button 622 on either side as shown in FIG. 24A. Snap features 690 can extend over the outer rim 670 of retention frame 604 and nest in corresponding snap recesses 692. Unlike the primary latching elements 622/662, which require deliberate user actuation of release button 622 and are depicted in FIG. 24B, passive snap elements 690 engage automatically when pump 602 is installed onto tray 604 and resist disengagement without requiring specific user manipulation.

Referring to FIGS. 24C-24F, snap elements 690 each include a projection 694 that extends inwardly towards retention frame 604. Projections 694 nest within snap recesses 692 and abut against downwardly facing retention surfaces 671 of the outer rim 670 of retention frame 604. Snap elements 690 do not directly move in response to movement of the release button 622, so when the release button is pressed to disengage the clips 662 from the retention frame 604, the passive snap elements 690 remain engaged and require an additional applied (upward) separation force to remove the pump 602 from the frame. Similarly, when the pump 602 is inserted onto the retention frame 604 an additional downward force is required to seat the snap elements 690 beneath the retention surfaces 671 of the outer rim 670 of the retention frame 604 following attachment of the clips 662.

The latch actuation interface of release button 622 can comprise a size-limited surface configured for intentional finger actuation, which reduces the likelihood that an incidental external load will selectively act on release button 622. However, when an external load accidentally actuates this primary latch, the geometry of the retention system—the size of the release button 622 and the proximity of the snap elements 690 to the release button—is configured such that the same external load will act on the snap features and, rather than disengaging those features, will increase engagement of the passive snap elements 690, thereby increasing the required separation force and maintaining pump attachment to the tray 604 in response to the accidental force. In other words, an accidental inward force on the release button 622 will also likely provide an inward force on the top surface 603 of the pump 602 which will cause the snap features 690 to be more firmly seated within recesses 692 to retain the pump 602 on the retention frame 604. An intentional disengagement by the user can still be easily accomplished with a combination of an inward force on release button 622 to release latches 662 and an upward force to disengage snap features 690.

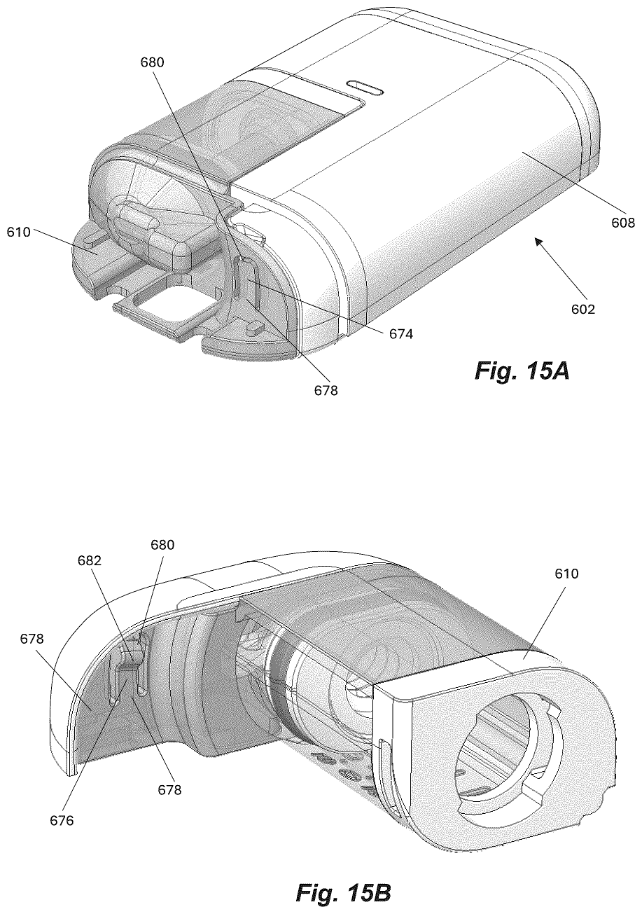

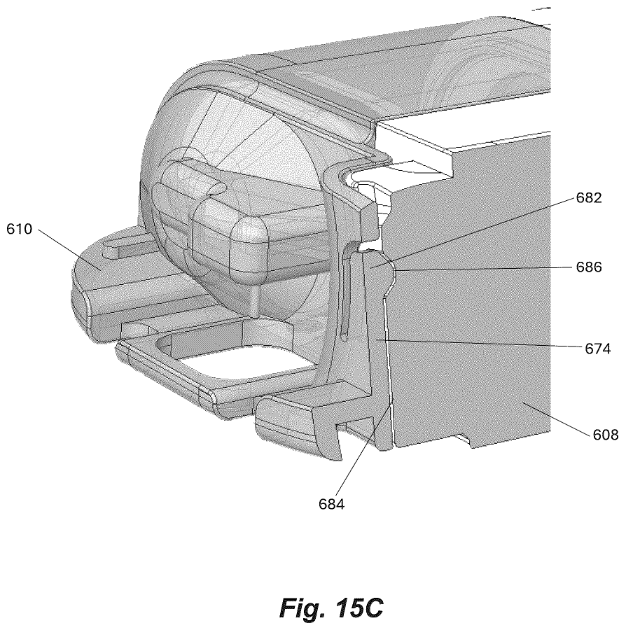

FIGS. 15A-15C depict aspects of the connection between the cartridge 610 and the drive unit 608 of the pump 602. Cartridge 610 can include a flexible arm 674 on an inner surface 676 thereof. Arm 674 can be cantilevered from a base end 678 and terminate at a tip 680 having a projection 682 extending inwardly therefrom. End 684 of drive unit 608 can include a recess 686 corresponding to the projection 682. As discussed above, cartridge 610 can be rotatably attached to drive unit 608. As the cartridge 610 is rotated, the arm 674 can flex upwardly from the base end 678 with the projection 682 interfacing with the end 684 of the drive unit 608. When the cartridge 610 is rotated such that the projection 682 is in alignment with the recess 606 in base 684, the arm 674 will snap down such that the projection 682 nests within the recess 686. This will provide haptic feedback to the user that the cartridge 610 is properly connected to the base 608. Once seated within recess 686, the projection 682 also serves in retaining the cartridge 610 on the drive unit 608 and preventing inadvertent detachment.

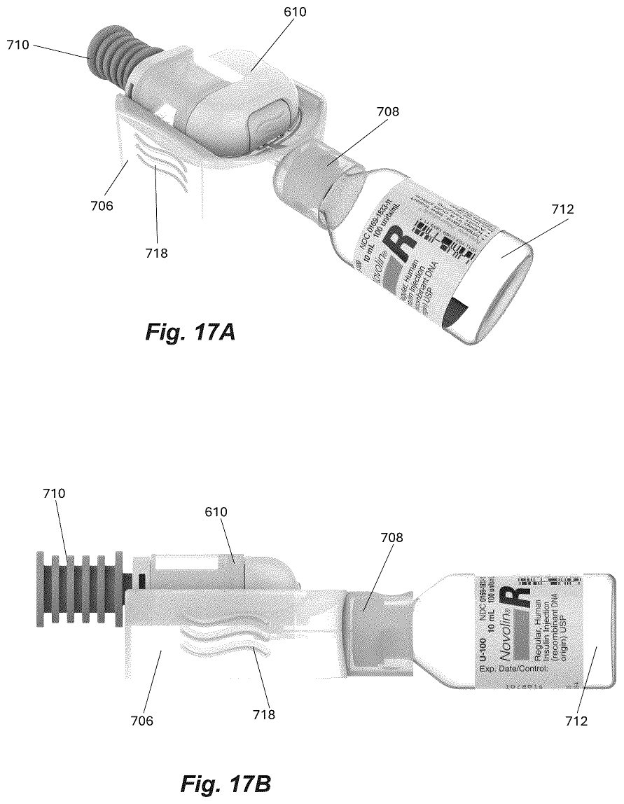

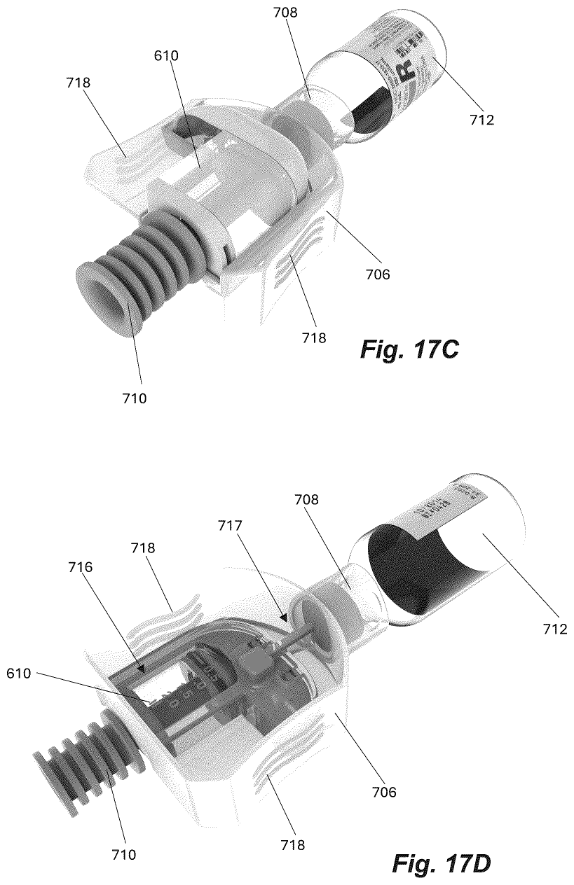

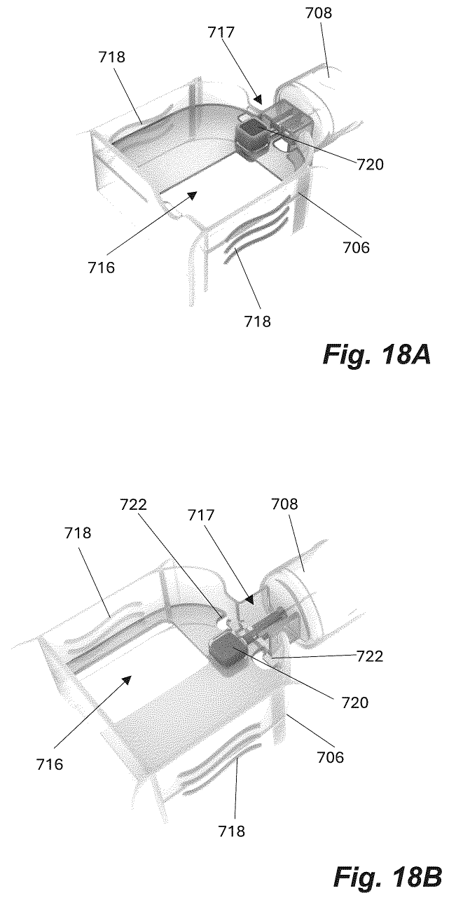

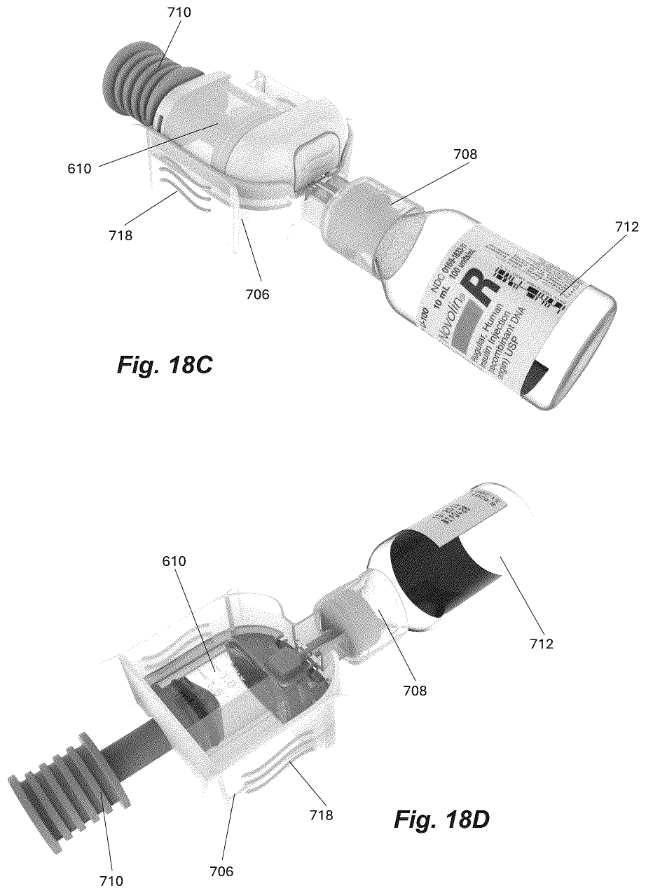

FIGS. 17A-17D and 18A-18D depict embodiments of fill adapters 706 that can be used to aid in filling the cartridge 610 of pump 602 with medicament. The embodiment of FIGS. 17A-17D is slightly larger which may make the device more ergonomic and easier to handle, whereas the embodiment of FIGS. 18A-18D is smaller and more streamlined to use less material and reduce waste.

Fill adapter 706 is configured to receive the cartridge therein 610. In this embodiment, vial adapter 708 is unitarily formed with the fill adapter 706 as a monolithic construct and facilitates a fluid connection between the cartridge 610 and a medicament vial 712. A septum interface 720 including a septum therein, most clearly seen in FIGS. 18A-18B, can fluidly connect the medicament vial 712 and the cartridge 610. Fill rod 710 can be attached to an opposite end of cartridge 610 from vial 712. The filling process is substantially similar to that described above with respect to FIGS. 4A-4D. In addition to viewing aperture 716 over the cartridge 610 that enables a user to visually monitor the filling of insulin in the cartridge from either side of fill adapter 706 (e.g., to monitor and enabling clearing of any bubbles in the insulin), the fill adapter 706 and/or vial adapter 708 can also be transparent in an area 717 adjacent to where the medicament vial 712 connects to the cartridge 610 to also enable a user to monitor the flow of insulin from the medicament vial 712. In this regard, it should be noted that while the upper surface of reservoir of cartridge 610 shown in FIG. 17C has a textured/semi-opaque surface, the lower surface shown in FIG. 17D is fully translucent. This dual-surface appearance approach can provide a desired appearance for the front of pump 102 as discussed above with respect to FIG. 16 while also enhancing the filling procedure by providing a clear view to the user during filling regardless of the appearance of the front surface of the reservoir. Fill adapter 706 can also include projections 718, which in this embodiment are configured as waves, that can aid in gripping the fill adapter during the filling procedure. In some embodiments, a filter can be added in the flow path between the vial 712 and the cartridge 610 to filter the insulin during the filling process. Fill adapter 706 may also include retention features 722 that can interface with corresponding features on the cartridge 610 to releasably lock the cartridge 610 onto the fill adapter 706 in a similar manner to how the cartridge 620 interfaces with the pump holder 604.

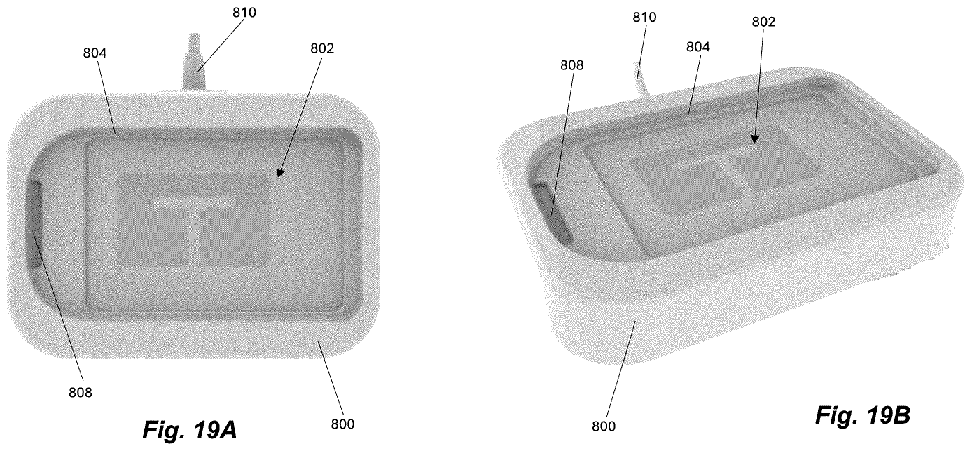

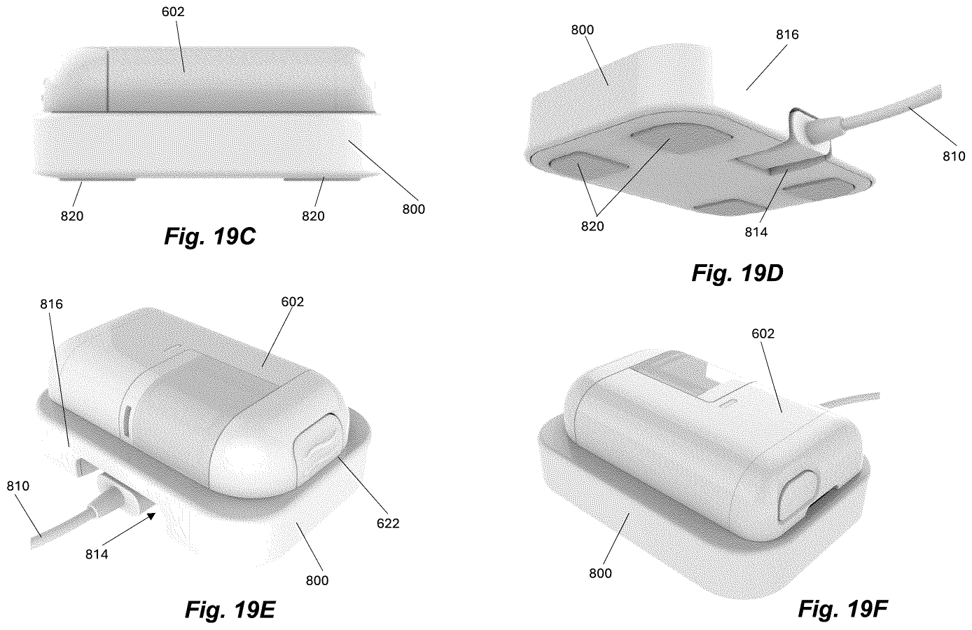

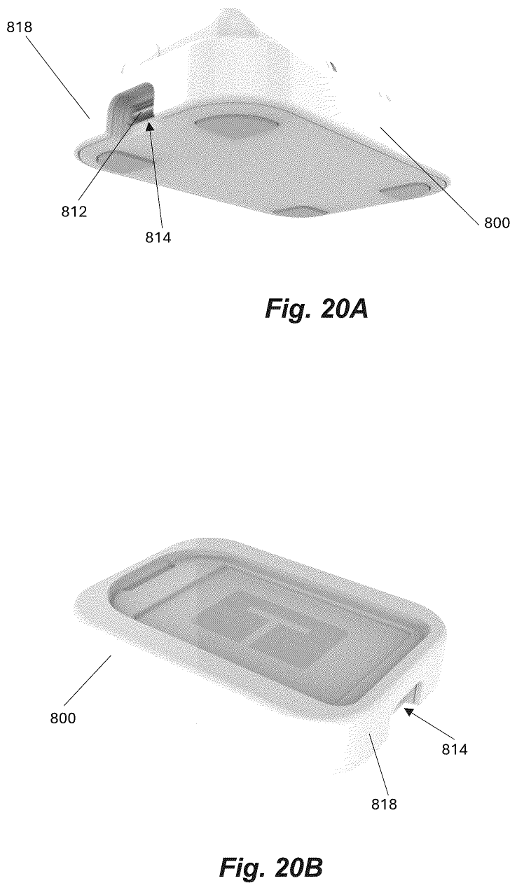

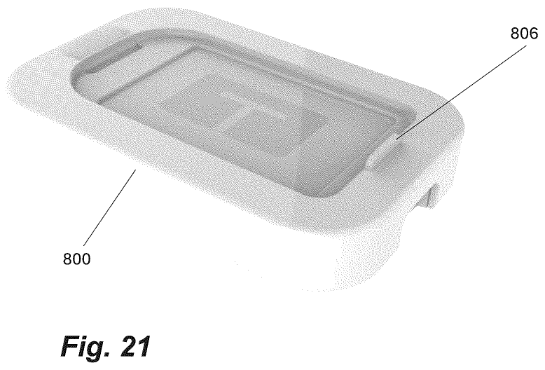

FIGS. 19A-19E depict an example of an inductive charging device 800 that can be utilized to charge the rechargeable battery of pump 602. Charger 800 has a more rectangular shaped design in comparison to the circular charger 500 of FIGS. 9A-9B. Charger 800 includes a recess 802 to receive and align the pump with the charging coil within charger 800 and a wall 804 laterally constraining the pump 602 on the charger. A locking recess 808 can be provided to interface with the clips 662 on the release button 622 of cartridge 610 to releasably lock the pump 602 onto the charger 800. Release button 622 can also be pressed to disengage clips 662 from the locking recess 808 to remove the pump 602 from the charger. In some embodiments, as depicted in FIG. 21, a latch 806 can be provided at an opposite end of recess 802 from locking recess 808 to further aid in interlocking pump in a manner similar to that described above with respect to FIGS. 9A-9B. A connector 812 for connecting the charger 800 to a power cord 810 can be provided in a recess 814 in a side 816 of charger 800. Alternatively, as depicted in FIGS. 20A-20B, the connector 812 and recess 814 can be provided on an end 818 of charger 800. In other embodiments, connector 812 may not be recessed. Grip pads 820 can be disposed on a bottom of charger 800 to aid in keeping charger 800 in place on a surface. In other embodiments, a charger such as charger 800 can be configured as a portable charger that does need to be plugged in to provide charging power to pump.

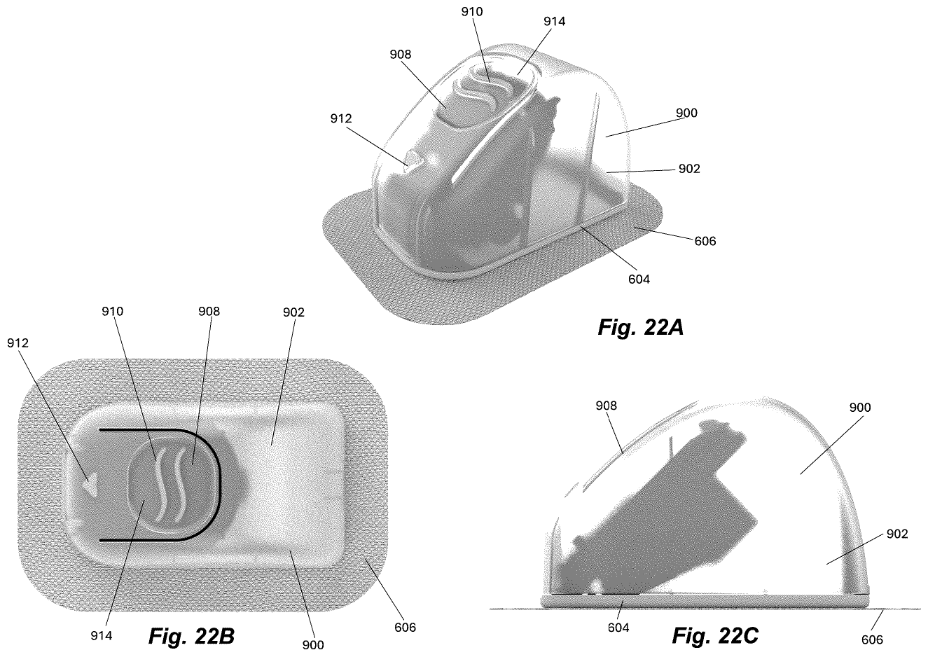

FIGS. 22A-22C depict an embodiment of an inserter 900 that can attach adhesive patch 606 and tray for holding pump 602 onto the body of the user and insert a cannula through tray 604 into the body of the user. Inserter 900 includes a housing 902 having an actuation button 908 therein that is actuated by pressing down on the button. After the user peels an adhesive backing off of the patch 606 and places the patch 606 on the user's body, the user can actuate button 908 to cause the inserter to generally simultaneously insert the cannula into the user's body and release the tray 604 from engagement with the inserter 900. The inserter 900 can then be discarded.

In this embodiment, actuation button 908 includes a pair of raised projections 910, which may be configured as waves, that aid in gripping the button and provide tactile feedback to indicate to the user that the user is pressing in the correct location on housing 902. Raised projections 910 can be located in a recessed trigger portion 914 of actuation button 908 that further provides a tactile and visual indication to the user of where to actuate the button 908. A positional indicator 912 can also be disposed on housing. Positional indicator 912 can be positioned generally adjacent to an area where the cannula will be inserted to aid the user in locating the inserter to insert the cannula in a desired location. In the depicted embodiment, inserter housing 902 is semitransparent. In other embodiments, housing 902 can be fully opaque or fully transparent. In the depicted embodiment, a bottom surface of the housing 902 contacts an upper surface of the outer wall of tray 604. In other embodiments, bottom of housing 902 may be wider to overlap and form a skirt around tray 604.

In embodiments, a patch pump system can include an adhesive patch configured to be adhered to a body of a user a retention frame disposed on the adhesive patch and an infusion pump configured to be releasably inserted onto the retention frame and including a reservoir configured to contain a medicament. A portion of the reservoir that is visible when the infusion pump is inserted onto the retention frame can be at least partially opaque (e.g., opaque or semi-transparent) and a surface of the reservoir that is not visible when the infusion pump is inserted onto the retention frame can be transparent to enable viewing of an amount of the medicament in the reservoir.

In some embodiments, the portion of the reservoir that is visible when the infusion pump is inserted onto the retention frame comprises a planar top surface and a rounded side surface.

In some embodiments, the surface of the reservoir that is not visible when the infusion pump is inserted onto the retention frame is a bottom surface opposite the planar top surface.

In some embodiments, the surface of the reservoir that is not visible when the infusion pump is inserted onto the retention frame comprises graduated markings to enable the user to visually gauge the amount of the medicament in the reservoir.

In some embodiments, the surface of the reservoir that is not visible when the infusion pump is inserted onto the retention frame is sufficiently transparent to enable a user to detect air bubbles in the medicament in the reservoir.

In some embodiments, the system further includes a fill adapter configured to receive the reservoir to enable filling of the reservoir with the medicament and the fill adapter includes a viewing aperture therethrough to enable a user to visually see the surface of the reservoir that is not visible when the infusion pump is inserted onto the retention frame during filling.

In some embodiments, the portion of the reservoir that is visible when the infusion pump is inserted onto the retention frame comprises a matte texture that renders the portion opaque.

In some embodiments, the matte texture is formed by texturing of a mold used in an injection molding process of the reservoir.

In some embodiments, the reservoir is formed from a resin comprising a colorant that achieves partial transparency of the reservoir.

In some embodiments, the portion of the reservoir that is visible when the infusion pump is inserted onto the retention frame is provided with a matte texture to increase the opacity of the partially transparent resin and the surface of the reservoir that is not visible when the infusion pump is inserted onto the retention frame is polished to increase the transparency of the partially transparent resin.

In embodiments, a patch pump system can include an adhesive patch configured to be adhered to a body of a user, a retention frame disposed on the adhesive patch, the retention frame including an outer rim and an infusion pump configured to be releasably attached to the retention frame. The infusion pump can include a release button including one or more clips configured to nest beneath a lower surface of the outer rim of the retention frame when the infusion pump is attached to the retention frame. The release button can be configured to be pushed inwardly to disengage the one or more clips from the retention frame. The infusion pump can further include one or more snap elements configured to extend over the outer rim of the retention frame, the snap elements configured to maintain engagement of the infusion pump with the retention frame when the release button is pushed inwardly.

In some embodiments, a force applied inwardly on the release button causes the one or more snap elements to increase engagement with the retention frame.

In some embodiments, when the release button is pushed inwardly, the one or more snap elements are configured to be disengaged from the retention frame by an upward force on the infusion pump relative to the retention frame.

In some embodiments, the one or more snap elements comprise a pair of snap elements, the pair of snap elements disposed on opposing sides of the release button.

In some embodiments, the one or more clips comprise a pair of clips with each of the pair of clips disposed adjacent to a corresponding one of the pair of snap elements.

In some embodiments, the release button is recessed within a shroud on an end of the infusion pump.

In some embodiments, the release button comprises one or more projections on an outer surface thereof configured to provide a gripping surface and a tactile indication to the user that the user is contacting the release button.

In some embodiments, the projections are configured as waves.

In some embodiments, the one or more snap elements are positioned adjacent to the release button such that an external load acting on the release button also acts on the one or more snap elements.

In some embodiments, the infusion pump is configured to be attached to the retention frame by inserting a first end of the infusion pump onto a hook on the retention frame and pivoting the infusion pump downwardly toward the retention frame to engage a second end of the infusion pump opposite of the first end including the one or more clips and the one or more snap elements with the outer rim of the retention frame.

Although the embodiments herein are specifically described with respect to the delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the foregoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A patch pump system, comprising:
an adhesive patch configured to be adhered to a body of a user;
a retention frame disposed on the adhesive patch; and
an infusion pump configured to be releasably inserted onto the retention frame and including a reservoir configured to contain a medicament, wherein:
a portion of the reservoir that is visible when the infusion pump is inserted onto the retention frame is at least partially opaque; and
a surface of the reservoir that is not visible when the infusion pump is inserted onto the retention frame is transparent to enable viewing of an amount of the medicament in the reservoir.

2. The patch pump system of claim 1, wherein the portion of the reservoir that is visible when the infusion pump is inserted onto the retention frame comprises a top surface and a side surface.

3. The patch pump system of claim 2, wherein the surface of the reservoir that is not visible when the infusion pump is inserted onto the retention frame is a bottom surface opposite the planar top surface.

4. The patch pump system of claim 1, wherein the surface of the reservoir that is not visible when the infusion pump is inserted onto the retention frame comprises graduated markings to enable the user to visually gauge the amount of the medicament in the reservoir.

5. The patch pump system of claim 1, wherein the surface of the reservoir that is not visible when the infusion pump is inserted onto the retention frame is sufficiently transparent to enable a user to detect air bubbles in the medicament in the reservoir.

6. The patch pump system of claim 1, further comprising a fill adapter configured to receive the reservoir to enable filling of the reservoir with the medicament, wherein the fill adapter includes a viewing aperture therethrough to enable a user to visually see the surface of the reservoir that is not visible when the infusion pump is inserted onto the retention frame during filling.

7. The patch pump system of claim 1, wherein the portion of the reservoir that is visible when the infusion pump is inserted onto the retention frame comprises a matte texture that renders the portion at least partially opaque.

8. The patch pump system of claim 7, wherein the matte texture is formed by texturing of a mold used in an injection molding process of the reservoir.

9. The patch pump system of claim 1, wherein the reservoir is formed from a resin comprising a colorant that achieves partial transparency of the reservoir.

10. The patch pump system of claim 9, wherein the portion of the reservoir that is visible when the infusion pump is inserted onto the retention frame is provided with a matte texture to increase the opacity of the partially transparent resin and the surface of the reservoir that is not visible when the infusion pump is inserted onto the retention frame is polished to increase the transparency of the partially transparent resin.

11. A patch pump system, comprising:
an adhesive patch configured to be adhered to a body of a user;
a retention frame disposed on the adhesive patch, the retention frame including an outer rim; and
an infusion pump configured to be releasably attached to the retention frame, the infusion pump including:
a release button including one or more clips configured to nest with the outer rim of the retention frame when the infusion pump is attached to the retention frame, the release button configured to be pushed inwardly to disengage the one or more clips from the retention frame; and
one or more snap elements configured to extend over the outer rim of the retention frame, the snap elements configured to maintain engagement of the infusion pump with the retention frame when the release button is pushed inwardly.

12. The patch pump system of claim 11, wherein a force applied inwardly on the release button causes the one or more snap elements to increase engagement with the retention frame.

13. The patch pump system of claim 11, wherein, when the release button is pushed inwardly, the one or more snap elements are configured to be disengaged from the retention frame by an upward force on the infusion pump relative to the retention frame.

14. The patch pump system of claim 11, wherein the one or more snap elements comprise a pair of snap elements, the pair of snap elements disposed on opposing sides of the release button.

15. The patch pump system of claim 14, wherein the one or more clips comprise a pair of clips with each of the pair of clips disposed adjacent to a corresponding one of the pair of snap elements.

16. The patch pump system of claim 11, wherein the release button is recessed within a shroud on an end of the infusion pump.

17. The patch pump system of claim 11, wherein the release button comprises one or more projections on an outer surface thereof configured to provide a gripping surface and a tactile indication to the user that the user is contacting the release button.

18. The patch pump system of claim 17, wherein the projections are configured as waves.

19. The patch pump system of claim 11, wherein the one or more snap elements are positioned adjacent to the release button such that an external load acting on the release button also acts on the one or more snap elements.

20. The patch pump system of claim 11, wherein the infusion pump is configured to be attached to the retention frame by inserting a first end of the infusion pump onto a hook on the retention frame and pivoting the infusion pump downwardly toward the retention frame to engage a second end of the infusion pump opposite of the first end including the one or more clips and the one or more snap elements with the outer rim of the retention frame.
